(19) United States
(12) Patent Application Publication
(10) Pub. No.: US 2020/0118689 A1
(43) Pub. Date: Apr. 16, 2020

(54) Fall Risk Scoring System and Method

(71) Applicant: Interactive Digital Solutions, Inc., Noblesville, IN (US)

(72) Inventors: Nicholas Luthy, Noblesville, IN (US); Sabastian Ricardo Diaz, S.C. Bariloche (AR); Brick Thompson, Noblesville, IN (US); Alex McDonald-Smith, Whitestown, IN (US); Alex Lindsay, Fishers, IN (US); Akshay Manohar Reddy, Fishers, IN (US)

(73) Assignee: Interactive Digital Solutions

(21) Appl. No.: 16/654,916

(22) Filed: Oct. 16, 2019

Related U.S. Application Data
(60) Provisional application No. 62/746,350, filed on Oct. 16, 2018.

(51) Int. Cl. G16H 50/30; G16H 10/60; G16H 15/00; G16H 80/00; G06N 20/00; G06F 3/0484

(52) U.S. Cl. CPC G16H 50/30; G16H 10/60; G06F 3/04847; G16H 80/00; G06N 20/00; G16H 15/00

(57) ABSTRACT

A system to evaluate the fall risk facing a patient and then adjust the care or observation toward that patient to minimize potential injury is provided. The system includes a fall risk scoring system which is based upon data collected from multiple inputs, including in-room data collection means and historical information. These scores are then updated continually and tracked, so additional knowledge and actions can be taken. Based upon the current scores, and historical scores, the system can then direct various adjustable and specific actions, with the goal of protecting the patient and minimizing injuries.

### CROSS-REFERENCE TO RELATED APPLICATIONS

This application claims the benefit of U.S. Provisional Application No. 62/746,350, filed Oct. 16, 2018.

### STATEMENT REGARDING FEDERAL SPONSORED RESEARCH OR DEV

Not applicable.

### Fall Risk Scoring System and Method

### Field of the Disclosure

The present disclosure relates to a fall risk scoring system and method to be used mainly in the health care field. Any number of issues can cause someone to face the risk of falling and the concurrent injuries that can result. The present disclosure is a system and method to evaluate the fall risk facing a patient and then adjusting the care or observation toward that patient to minimize potential injury.

### BACKGROUND OF THE INVENTION

Fall prevention is something that providers of both inpatient and remote health-care put at the forefront of their concerns about certain patients. This is particularly true for those patients that either have a condition which impacts stability or mobility or perhaps just due to age. Thus, they often classify certain individuals as being at a risk to fall or at a risk to suffer serious injury if they were to fall. However, preventing falls and providing effective, immediate assistance after every fall can be difficult, if not impossible. This may be even more true when patients are monitored by staff that is responsible for observing or managing multiple patients. Thus, people responsible for this care seek to minimize the risk of falling or injury, as much as possible. Historically, the means used to lessen fall risks was to station a person in the proximity of the individual to be observed. However, this is a costly solution and generally inefficient solution.

Therefore, technology has developed that is centered on fall-detecting devices. Typically, such devices monitor certain activity of the patient and then informs a provider when the patient is undertaking some prescribed activity. In response to this information, the provider can theoretically then provide better assistance.

U.S. Pat. Nos. 6,611,783 and 7,127,370 disclose one such monitoring system. This system includes a monitoring device which tracks a patient's body position and detects when the patient is attempting to stand. When the monitoring device detects that the patient is attempting to stand, it triggers an audible and visible alert, perceivable by the patient and one or more providers, to assist the patient. There are also other devices offered by various companies that use various means to identify patient activities and to inform the patient and others that the patient is engaging in fall-related behavior.

Further, U.S. Pat. No. 7,612,681 discloses another monitoring system. This system incorporates radar sensors to detect movement by the patient though the home. This system uses these sensors to detect location and movement, but can also evaluate walking features, such as gait speed, gait length, variable movement speed and gait/balance instability, among other things. However, no use of video or audio data is incorporated into this system.

However, each of the monitoring systems described above does not address certain issues or have shortcomings. First, the devices are either required to be strapped or attached to the patient in some way, or be within contact of the patient, such a pressure pad or other device. These restrict, or make the patient uncomfortable, thus impairing their ability to properly rest or heal. Second, none of the inventions incorporate the use of video or audio data to create a fall-risk score. They normally only provide an immediate audible or visible alarm and they are not able to collect sufficiently precise data (more than simply location and movement) to accurately assess the fall risk of a patient.

Accordingly, a system and method is needed that does not require a patient to have another device or machine attached to them or within their vicinity, and one which will take real-time video and/or audio data, including historical information, to create an accurate fall-risk score which will allow providers to minimize or eliminate fall risks. There is also a need for a system and method to allow for simultaneous management of multiple patients by a single or limited number of providers.

### BRIEF SUMMARY OF THE INVENTION

To this end, the subject of this invention is a system and method for evaluating the fall risks an individual might be facing by evaluating cues and subtleties from video and/or audio data, without the need for attachment of a device to the patient or for certain equipment being within close proximity of the patient, to determine when an individual may need a higher level of care than previously established.

This system and method includes one or more of the following elements: a means to obtain video data and audio data from a room or area where the patient (collectively referred to hereinafter as “Patient”) is located. This data, either separately or together, is transmitted to a location (either physically or digitally) for additional consideration and/or processing. There would also be a means for an observer or medical provider (hereafter referred to as “Observer”) to monitor the video and/or audio data, or other data derived from additional sources, including historical information about a Patient, and then interact with a user interface (“UI”), which would allow the Observer to make notes or document what is happening and/or to take certain actions as necessary or be directed to take certain actions. There could also be a means for the Observer to monitor multiple feeds of audio or video data from multiple different Patients using the UI. Likewise, there could also be a means to obtain video or audio data on the Observer. There could likewise be a means of communication (potentially by video, audio, or digitally) between the Observer and the Patient, as well as between the Observer and third parties and also, between the Patient and other third-parties.

Based upon all of the data collected, including historical information about the Patient, it can be processed into an accurate fall-risk score, which could then dictate either a certain level of care and/or certain safeguards be put in place to raise the level of service to the Patient and/or certain actions be taken to address Patient needs.

### BRIEF DESCRIPTION OF THE DRAWINGS

FIG. 1 is a diagram of one embodiment of the data collection and treatment process of the method of this invention.

### DESCRIPTION

For the purpose of promoting an understanding of the principles of the present invention, reference will now be made to the embodiment illustrated in specific language contained herein. It will, nevertheless, be understood that no limitation of the scope of the invention is thereby intended; any alterations and further modifications of the described or illustrated embodiments and any further applications of the principles of the invention as illustrated therein are contemplated as would normally occur to one skilled in the art to which the invention relates.

The preferred embodiment of the method and system in this disclosure would one that incorporates all the referenced features, including both video and audio data, however, it should be noted that a method or system using either only video data or audio data would also be effective.

This system and method would incorporate one or more of the well-recognized means to collect and transmit video and/or audio data in digital format. Given this requirement, the elements of the preferred embodiment of the system indicated herein, can be broken down as follows, with the following potential capabilities as explained below—video data, camera control, audio data and UI.

Video Data

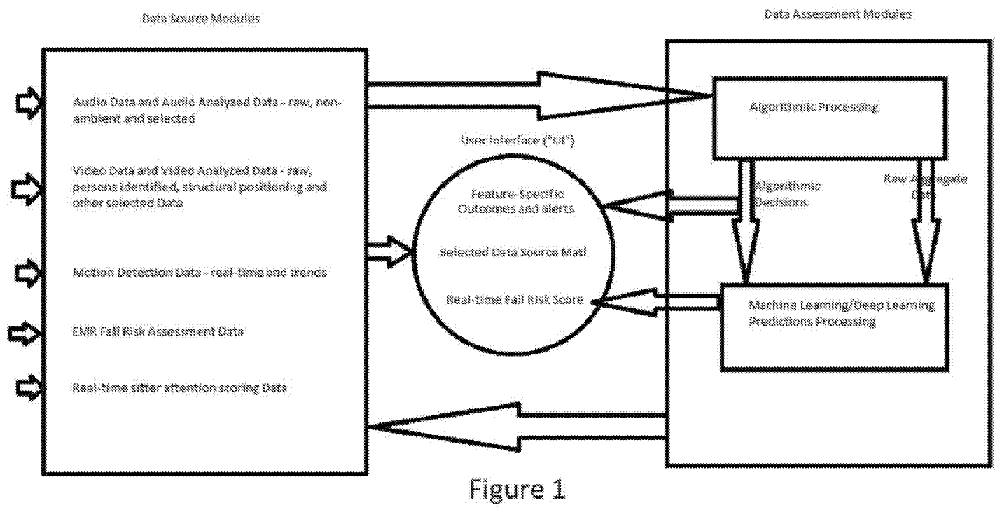

Once obtained in digital format, the video data would be transmitted to a data source module, see FIG. 1, for collection and initial processing. This initial processing could include in video data—person identification, structural positioning of the patient within the room, and motion detection processing; for audio data—this could include stripping ambient noise and selected other audio elements. This area would also receive information from electronic medical record (“EMR”) data sources as to the Patient or Patients involved, including any initial fall-risk scoring.

As noted above, the video data can be processed to examine motion detection in the realm of both real-time triggers above a threshold, as well as a trending analysis over time, such that guidance can be provided to the Observer to focus on an awake Patient or a Patient that has switched the trend (gone from sleeping and low motion to awake and more consistent motion regardless of degree of motion), as well as other measures.

An additional data treatment of the video data would be the video contexts. This would include processing the video data to determine how many people are in the area, whether the people are sitting or standing (and more specifically the Patient). Based on this information and other data, a determination could be made as to the level of attention to the Patient that is needed during a specific period.

Both of these elements of video data are then combined with other data and additional historical data from the Patient regarding past falls or conditions that contribute to falling, etc. and is then sent to a Data Assessment Module for additional processing, which could include algorithmic processing, after which machine learning and deep learning processes are applied to create an initial real-time fall-risk score for the Patient. Based upon the score, certain actions are taken to either or both, dictate a certain level of care and/or safeguards to assist the Patient. At the same time, the initial fall-risk score is returned to the data source module for consideration in light of other real-time data, to calculate second and subsequent fall-risk scores, all of which are saved and could be viewed as a trend.

A further embodiment of the method and system could include video data related to the Observer using this system. This could include capabilities regarding motion detection and facial recognition in order to develop a proprietary attentiveness score for the Observer. If it falls below a certain pre-determined standard, then other feedback can be provided to the Observer to increase their attentiveness and/or an alert can be made to a third-party, or other means can be taken to increase attentiveness of the Observer.

Another embodiment of this system could also provide for increased accuracy using real-time facial recognition, with could be applied to the Patient and/or the Observer, with density weighted motion detection. This is done by taking video data from a Patient or Observer and deconstructing it to its individual frames. Then this invention would compare frame n with n−1 (current to the previous) for a pixel-by-pixel differential. The pixels that have changed are determined to be caused by motion of some degree (human motion or change of vantage point—i.e. someone moved the camera). Then the system applies a weighted measure to the density (how compact the differentials are) to:
i. Determine false positive movement from that of true movement. If a high percentage of pixels have changed and there is no centralized density, it is likely that someone has moved the camera, or that there are multiple people in the area, but in either case, it would not an event in which action is necessarily required.
ii. With a density of pixel differential (i.e. motion), we can focus our person identification and facial identification algorithms on those areas specifically, thus reducing the processing power required. (e.g. if the pixels differed only in 15% of the screen, then we only look at those areas of the frame for “people” or “faces”). This will also eliminate false positive results when we enable our ability for a camera to auto-track the Patient's face.

An additional element incorporating the video data could be potential to pull still photos from the video data, at certain prescribed times, or upon the direction of the Observer. Then post-processing of these photos could be undertaken by the system to determine in-room situational awareness. For example, when the Observer engages with a Patient in order to verbally redirect them, the system automatically captures a photo of the before and after engagement by the Observer. The system will then do a post-processing analysis to determine common components in the room or area with a specific focus on people (Patient, physician, nurse, tech, or visitor). The system may gather feedback from the Observer to increase accuracy of the algorithms employed by the system. The goal would be to provide details and analytics to the organization employing the system, particularly when an incident occurs in the room or area.

In addition to the video data, an additional source of data, as mentioned above, which could be applied to the video, audio or other processes indicated herein, could be information derived from an EMR system about the Patient. This information could be used to evaluate other data (video, audio or other) being seen and to also determine whether this other data should be considered differently. In some cases, this data could provide a baseline, which is then further enhanced by the real-time video data and also analyze or process that the system might perform.

Camera Control

In most instances, the video and potentially audio data will be obtained via a camera and this system provides for a multitude of ways to control the camera. For instance, the camera could be moved and controlled via the system manually, by auto-tracking, or on a smart basis (potentially using artificial intelligence), and by far-end camera controls.

For example, one mode could be auto-tracking of the Patient. In this mode, the system keeps track of how many faces are in the Patient's area. If there is only one face, presumably the Patient, then the system can be enabled to issue commands to the camera to reposition itself through panning, tilting, and zooming to follow the face of the Patient. This would enable a report of Patient observation, while keeping resources to a minimum, and a low touch mode while keeping the Patient in view. If the system were to determine there were multiple faces, the system will process through a series of decisions to determine which face is most likely to be the Patient's. An example of such a decision, but is not limited solely to this decision, are any of the faces identified within the frame also within a detected object, such as a bed, or near where the Patient has historically been found. A further means would be a determination to map out the body skeletal structure of the Patient, via machine learning models, to determine if a certain face is associated with the Patient, or if it is another person not under observation. This could be applied whether the Patient is in the bed, laying down, or a body seated near the bed.

An alternative functionality of the system could be zoom-to-face. While this functionality is similar to the one noted above regarding making a determination of which face is associated to the Patient, however, in this case, rather than the system issuing automated commands to position the camera, the Observer can press a button which may trigger a more significant movement of the camera to an intelligent framing of the Patient's face. This could be used to determine if the Patient is in distress or is otherwise in need of assistance.

An additional embodiment of the system could be nearly universal Application Programming Interfaces (“API”) that allow the system to make common commands that are then parsed and translated into camera specific commands to make movement-capable cameras more intelligent. The capability can issue commands based upon the auto tracking, manual commands (as noted above), or future smart capabilities (which would most likely incorporate AI).

A further embodiment of the system would be the ability to enable far-end camera control from a remote browser to a local client that is also browser-based. In other words, the Observer's application interface being remote and the Patient's application interface being local. In this functionality, the command originates from either the Observer's UI, or the application's server and is sent to the Patient's client UI. The client UI then issues a command to a service on their local device, via an API, to broker the commands to the various enabled camera devices.

Audio Data

The digital audio data, on it most basic level, could simply be used to monitor the audio activity taking place in the area being observed. However, another embodiment of this system could be the consideration and examination of the audio data to identify non-ambient noise. This consideration would sonographically “print” the room or area (like fingerprints, but with audio) so that over time the standard noise can be “filtered out” to focus on non-ambient noise and determine when a spike in audio occurs, which may indicate the need for heightened attention or some action on behalf of the Observer.

An additional embodiment of the system could also be ability for the audio data from the Patient to be translated into whatever language is understandable to the Observer. Likewise, the audio data from the Observer could be translated into the language understandable to the Patient. This would be done on a real-time basis and would eliminate the need for translators or other third parties to be involved in the care of the Patient. A similar capability could also be done via text-to-speech that may be exchanged between the Patient and Observer. One option that could be used in this case is as follows:
a. Capture input text from the Observer in the form of a text box.
b. The system learns of the language disparity via medical record data or other means for the Patient and the settings data for the Observer.
c. The system automatically translates the text from the Observer's language to the Patient's language.
d. Then the translated text leverages a voice synthesis engine to convert it from text to speech.
e. The synthesized voice is the played to the Patient over the audio output (i.e. speaker) within the room.

User Interface or UI

The data and other information used by the system would be displayed and available to the Observer, or other parties, using a UI contained on a fixed or mobile display. One or more unique embodiments of the UI in the system could include the following:
a. For example, up to ten (“10”) persistent video/audio connections;
b. The ability of the Observer to focus on one of the ten (“10”) without blocking out the other connections;
c. The ability to not require a second session or call be established to communicate with the Patient;
d. The ability of the system to quickly contact an Observer via SMS (or similar means) or via voice. In this case, the voice doesn't require a physical phone, rather the system transfers the audio onto the Observer's software client and connects to a third-party (such as a nurse), or the Patient, over the Public Switched Telephone Network or PTSN.

Whatever amount of data is available, they would contribute to an overall score and scoring for a predictive fall analysis. Since the outcome is a real-time score, it also allows the monitoring of the trend to that score, which can be used to allow the Observer to be proactively alerted, or to automatically trigger response alerts.

The method and system is claimed as follows:

1. A method for determining the fall-risk potential of a patient comprising a host device configured to communicate with a plurality of data devices, each of the plurality of data devices providing data to the host device and wherein the host device is further configured to:
(a) store patient-specific identification data in conjunction with gathering data from each of the data devices associated with that patient;
(b) store the data from each device received in association with the patient;
(c) store historical patient-specific health and related data relevant to a fall-risk score;
(d) analyze the data from the devices and the historical data to create a fall-risk score for the patient, which is also stored;
(e) at a later time, continue to analyze the data from the devices and applicable historical data to create additional fall-risk scores, which are also stored;
(f) send the results of the analysis for additional processing to other modules; and
(g) send the results, either before and after additional processing, to a user-interface for the user to apply or interact with the results and to adjust patient care as necessary;
(h) continue the process starting with step (a) as additional data is gathered and additional adjustments are made.

2. A method of claim 1, wherein the data devices provide digital video and audio data.

3. A method of claim 1, wherein the additional processing includes algorithmic interpretation.

4. A method of claim 1, wherein the additional processing includes machine learning and deep learning predictions.

5. A System for monitoring patients comprising:
a) a means to control a data collection source which can then obtain digital data regarding a patient's activities and status and to send said data to a data source module and user interface;
b) a means to control a data collection source which can then obtain digital data regarding an observer's activities and status and to send said data to a data source module and user interface as well as third-parties;
c) a means to obtain digital data regarding a patient's historical health information and to send said data to a data source module and to also send it to a user interface for interaction;
d) a data source module containing various initial processing capabilities and a data assessment module, for more intense processing, including algorithmic and machine learning/deep learning processes, which results in a patient fall-risk score, and a means to send the results thereof to other processes contained in the modules and to a user interface;
e) a user interface for an observer to see data transmitted thereto and to interact with data and input data and document changes necessary to adjust patient care and treatment and to operate the system.

6. The System of claim 5 wherein the digital data consists of video and audio data.

7. The System of claim 5 wherein the control of the data collection source is related to a video camera and the controls allows manual movement, auto-tracking of persons, smart-tracking and zoom-to-face.

8. The System of claim 5 wherein the processing capabilities include the ability for motion detection and contextual analysis, with the ability to trigger certain actions given the results via the user interface, or for additional processing.

9. The System of claim 5 further comprising control of the data collection source by remote browser control or application programming interface (“API”) control for common commands.

10. The System of claim 5 further comprising a means of communication, including video, audio or digital, between the patient and/or the observer and/or third-parties.

11. The System of claim 5 further comprising control of the data collection source by remote browser control or application programming interface (“API”) control for common commands.

12. The System of claim 5 further comprising processing of video data related to the observer, including motion detection and facial recognition, to determine an attentiveness score, which could dictate additional actions by the observer or notice and action by third-parties.

13. The System of claim 5 further comprising processing of video data of both patients and observers using facial recognition with density weighted motion detection, to dictate additional actions by the observer or notice and action by third-parties.

14. The System of claim 5 further comprising the ability to generate still-photos from the video data either at certain prescribed times determined or programmed into the system or manually by the observer, which can then be saved or used for additional processing and determination of additional actions.

15. The System of claim 5 further comprising processing of audio data related to creating an audio profile of the area where the patient is located in order to filter out ambient noise so clearer attention can be given to the non-ambient noise in the area, in order to more easily hear when a patient has a medical need.

16. A System for monitoring patients comprising:
a) a means to observe multiple patients at the same time using one user interface with one observer;
b) a means to control a data collection source which can then obtain digital data regarding a patient's activities and status and to send said data to a data source module and user interface;
c) a means to control a data collection source which can then obtain digital data regarding an observer's activities and status and to send said data to a data source module and user interface as well as third-parties;
d) a means to obtain digital data regarding a patient's historical health information and to send said data to a data source module and to also send it to a user interface for interaction;
e) a data source module containing various initial processing capabilities and a data assessment module, for more intense processing, including algorithmic and machine learning/deep learning processes, which results in a patient fall-risk score, and a means to send the results thereof to other processes contained in the modules and to a user interface;
f) a user interface for an observer to see data transmitted thereto and to interact with data and input data and document changes necessary to adjust patient care and treatment and to operate the system.

17. The System of claim 16 wherein the means to observe multiple patients includes the ability to observe all of them at the same time or to zoom onto one patient, while still maintaining data streams to the user interface from the other patients.

18. The System of claim 16 wherein the digital data consists of video and audio data.

19. The System of claim 16 wherein the control of the data collection source is related to a video camera and the controls allows manual movement, auto-tracking of persons, smart-tracking and zoom-to-face.

20. The System of claim 16 wherein the processing capabilities include the ability for motion detection and contextual analysis, with the ability to trigger certain actions given the results via the user interface, or for additional processing.

21. The System of claim 16 further comprising control of the data collection source by remote browser control or application programming interface (“API”) control for common commands.

22. The System of claim 16 further comprising a means of communication, including video, audio or digital, between the patient and/or the observer and/or third-parties.

23. The System of claim 16 further comprising control of the data collection source by remote browser control or application programming interface (“API”) control for common commands.

24. The System of claim 16 further comprising processing of video data related to the observer, including motion detection and facial recognition, to determine an attentiveness score, which could dictate additional actions by the observer or notice and action by third-parties.

25. The System of claim 16 further comprising processing of video data of both patients and observers using facial recognition with density weighted motion detection, to dictate additional actions by the observer or notice and action by third-parties.

26. The System of claim 16 further comprising the ability to generate still-photos from the video data either at certain prescribed times determined or programmed into the system or manually by the observer, which can then be saved or used for additional processing and determination of additional actions.

27. The System of claim 16 further comprising processing of audio data related to creating an audio profile of the area where the patient is located in order to filter out ambient noise so clearer attention can be given to the non-ambient noise in the area, in order to more easily hear when a patient has a medical need.
